(19) United States
(12) Patent Application Publication
(10) Pub. No.: US 2006/0018936 A1
(43) Pub. Date: Jan. 26, 2006

(54) Core for cosmetic articles

(75) Inventors: Reiner Appel, Oberasbach (DE); Gerhard Lugert, Nurnberg (DE); Harald von Godin, Oberasbach (DE); Jachim Kinzel, Stein (DE)

Correspondence Address: VENABLE LLP, P.O. BOX 34385, WASHINGTON, DC, 20045-9998, US

(73) Assignee: Faber-Castell AG, Stein (DE)

(21) Appl. No.: 11/074,843

(22) Filed: Mar. 9, 2005

(30) Foreign Application Priority Data
Jul. 23, 2004 (DE) 20 2004 011 608.1
Aug. 12, 2004 (DE) 20 2004 012 652.4

(51) Int. Cl. A61K 8/36

(52) U.S. Cl. 424/401

(57) ABSTRACT

A cosmetic product core comprises a basic core mass and solid particles dispersed therein. The basic core mass includes at least one fatty acid salt and at least one lipophilic material with a melting temperature range<50° C.

### CROSS-REFERENCE TO RELATED APPLICATIONS

This application claims the priority of German Patent Application No. 20 2004 011 608.1, filed on Jul. 23, 2004 and German Patent Application No: 20 2004 012 652.4, filed on Aug. 12, 2004, the subject matter of each of which is expressly incorporated by reference herein.

### BACKGROUND OF THE INVENTION

The invention relates to cosmetic product cores used for decorative applications as well as skin-care purposes. Such cores are used in lipsticks, lip-liners, eye shadow products, eyeliners, cosmetic pencils, rouge sticks and the like. The cores of the invention may also take the form of wax sticks or pencils. In general, these cores contain a basic core mass, composed of wax-type substances such as waxes, fats, and emulsifying agents, as well as oils, filler materials and pigments.

The disadvantage of prior cores of this type is their relatively low temperature stability and, in particular, low maximum application temperature. The maximum application temperature in this case is understood to be the temperature at which the core mass or the wax stick mass can still be applied to the lip, the eyelid or other skin section without smudging, without deformation of the core, or breakage of the core and/or the wax stick. To be sure, traditional cores have melting points and/or melting ranges between 60° C. and 90° C., but the maximum application temperature is naturally much lower. The methods used to form the core influence the microstructure of the basic core mass and thus also the mechanical and application characteristics of the subsequently produced core. The microstructure is influenced least when using a molding technique which produces cores which allow later application temperatures of approximately 40° C. to 45° C. When using the extrusion technique, where the basic core mass is compressed inside a cylinder and leaves this cylinder in the form of a rope via an exit opening, the basic core mass is stressed mechanically and thermally to such a degree that the finished cores have application temperatures ranging only from approximately 30° C. to 35° C.

Starting with these facts, it is an object of the present invention to provide a core for cosmetic applications which shows improved temperature stability and, in particular, can be applied at higher temperatures.

### SUMMARY OF THE INVENTION

In accordance with the invention, there is provided a cosmetic product core comprising a basic core mass and solid particles dispersed therein, wherein the solid core mass comprises at least one fatty acid salt and at least one lipophilic substance with a melting range<50° C., meaning an oil or a wax-type substance. At least 50% of the basic core mass is comprised of the solid particles.

The basic core mass is understood to be a mass that functions as a bonding agent for the solid particles and which is primarily responsible for the application characteristic and the thermal stability of the core.

### DETAILED DESCRIPTION

Surprisingly, it has turned out that cores having a melting point above 90° C. can be produced with such basic masses, meaning cores which can survive far higher storage temperatures than traditional cores without suffering damage and which can be applied at higher temperatures, namely at temperature of more than 50° C. More surprisingly, is the fact that cores with the proposed basic core mass can absorb up to 90% solid particles, thus allowing the production of very color-intensive core preparations. Cores of this type cannot be produced with traditional basic core masses. As a result of the high proportion of solid particles, the viscosity of such a molten core mass would be excessively high and would prevent it from being poured. Moreover, rod-extruding techniques or the extrusion by screw extruder could also not be used with conventional basic core masses, since traditional basic core masses would be liquefied excessively at the necessary temperatures.

A core mass according to the invention, on the other hand, can be extruded without problems at temperatures ranging from approximately 80° C. to approximately 140° C., among other things because the core mass and/or the basic core mass itself still has sufficient viscosity during the processing, even at these temperatures. Particularly good results can be achieved with cores containing 1% to 20% and preferably 3% to 10% fatty acid salt, wherein the best results are achieved with a fatty acid content of 4% to 8%. The lipophilic substance should be present in shares of 5% to 40%, preferably 10% to 35%, wherein a lipophilic substance content of 16% to 32% has proven to be particularly suitable.

A solid particle content of at least 50%, preferably of 60% to 90%, has proven favorable for the temperature stability of the cores and particularly for the application temperature. Solid particles in this case are understood to be filler materials such as talcum and mica, but also color pigments.

Suitable fatty acid salts are those having chain lengths of 10 to 20 C-atoms, in particular calcium laurate, magnesium stearate, zinc stearate, but also palmitates, myristates, oleates and linoleates can be named in this connection. Particularly suitable are lithium stearate, aluminum stearate, aluminum tristearate, as well as mixtures of the aforementioned fatty acid salts, wherein optimum results are achieved with aluminum distearate.

The following can be used as lipophilic liquids and/or oils or waxy substances with a low melting point<50° C.: castor oil, mineral oil, stearyl dimethicone, hydrogenated (hardened) coconut oil, isopropyl palmitate, isopropyl myristate, triglycerides such as capryl and caprin triglycerides, hydrogenated palm-seed oil glycerides, and hydrogenated palm-oil glycerides, as well as olive oil, rapeseed oil and other oils generally used for cosmetic articles.

Organic or inorganic pigments generally used for cosmetic articles can be added as coloring agents. Also conceivable is the use of colorants such as lipophilic colorants. Filler materials such as talcum, kaolin, mica or mica coated with silicon dioxide can also be added if necessary. Preservatives, sunscreen agents, aromatic substances, vitamins, perfumes, healing substances, but also small amounts of structure-forming and/or texture forming substances such as C 30-45 alkyl methicone (CAS No: 131459-42-2) can be used as admixtures or auxiliary agents. The sum of all additives in the recipe should not exceed 10%.

The masses according to the invention are homogenized in mixers, kneaders, roller mills and the like and can be extruded at 80-140° C. to form cores or wax sticks.

### EXAMPLES

The following composition is given for the basic recipe:

| | |
|---|---|
| salt of a fatty acid | 1-20% |
| lipophilic substance | 5-40% |
| with a melting point <50° C.: | |
| pigments, filler materials: | 30-90% |
| additives: | 0-10% |

One preferred basic recipe has the following composition:

| | |
|---|---|
| salt of a fatty acid | 4-8% |
| lipophilic substance | 16-32% |
| solid material particles | 60-80% |
| Additives | 0-10% |

The invention is to be illustrated with the following concrete examples:

### Example 1

Blue eyeliner core with a core diameter of 4 mm:

| | |
|---|---|
| lithium stearate: (CAS 4485-12-5) | 8% |
| isopropyl myristate: (CAS 110-27-0) | 20.0% |
| hydrogenated palm-seed glycerides | 11.0% |
| (commercial name: “Lipocire CM;” can be | |
| obtained at: Gattefosse): | |
| pigment blue 27: CI 77510 | 60.0% |
| panthenol: (CAS 16485-10-2) | 0.5% |
| propylparaben: | 0.5% |

### Example 2

Recipe for solid black cosmetic article core, used for eye applications. The core has a diameter of 3 mm for applications to dry skin and moist skin (eyeliner and cosmetic pencil applications). The cores can be applied at temperatures of up to 60° C., wherein the temperature stability is higher than 95° C.

| | |
|---|---|
| aluminum distearate (CAS 300-92-5): | 4.0% |
| caprylic/capric triglycerides: | 16.0% |
| (commercial name: Myritol 318; | |
| supplier/producer: Henkel) | |
| pigment blue 29 CI 77007 | 5.0% |
| mica coated with silicon dioxide | 15.0% |
| (commercial name: Micronasphere M | |
| (Merck) | |
| pigment black 11; CI 77499 | 60.0% |

### Example 3

Wax stick with a diameter of 10 mm for metallic, glossy skin markings, having a maximum application temperature of 55° C.:

| | |
|---|---|
| aluminum stearate (CAS 7047-84-9) | 4.0% |
| zinc stearate (CAS 557-05-1): | 2.0% |
| hydrogenated palm oil/palm-seed oil | 10.0% |
| PEG-6-ester: | 10.0% |
| (commercial name: Lipocire BP Pastilles | |
| manufacturer/supplier: Gattefosse) | |
| hydrogenated coconut oil (CAS 84836-98-6) | 12.0% |
| talcum: | 20.0% |
| aluminum powder: pigment metal 1; CI 77000 | 42.0% |

### Example 4

Recipe for dark red lipstick core, 8.0 mm diameter, for applying a dark-red non-glossy color, said core having a melting point of >90° C.:

| | |
|---|---|
| aluminumdistearate: | 4.0% |
| stearyldimethicone | 16.0% |
| (commercial name: Dowcorning 2503; | |
| manufacturer: Dowcorning) | |
| propylparaben: | 1.0% |
| talcum: | 28.0% |
| natural red 4; CI 75470 | 51.0% |

### Example 5

Core recipe with weak coloring and containing healing earth (loess), for skin applications. The core diameter is 5 mm for applications on dry or moist skin. The cores can be applied at temperatures up to 60° C. and have a temperature stability of more than 95° C.

| | |
|---|---|
| aluminumdistearate (CAS 300-92-5): | 4.0% |
| caprylic/capric triglycerides: | 16.0% |
| (commercial name: Myritol 318; | |
| supplier/manufacturer: Henkel) | |
| healing earth (loess), ground (D90 ≦ 20 μm) | 50% |
| hydrogenated palm-seed glycerides | 5% |
| (commercial name: Lipocire CM; | |
| can be obtained from: Gattefosse): | |
| mica coated with silicon dioxide | 24.0% |
| (commercial name: Micronasphere M (Merck) | |
| propylparaben: | 1.0% |

All percentages listed are weight percentages.

The invention has been described in detail with respect to preferred embodiments, and it will now be apparent from the foregoing to those skilled in the art, that changes and modifications may be made without departing from the invention in its broader aspects, and the invention, therefore, as defined in the appended claims, is intended to cover all such changes and modifications that fall within the true spirit of the invention.

What is claimed is:

1. A cosmetic product core comprising:
a basic core mass and solid particles dispersed therein,
wherein the basic core mass comprises:
at least one fatty acid salt; and
at least one lipophilic material with a melting temperature range<50° C. and wherein the cosmetic product core comprises at least 50 weight % of said solid particles.

2. The cosmetic product core of claim 1, wherein the basic core mass has a fatty acid salt content which ranges from 1% to 20 weight %.

3. The cosmetic product core of claim 1, wherein the basic core mass has a fatty acid salt content which ranges from 3% to 10 weight %.

4. The cosmetic product core as defined in claim 2, wherein the basic core mass has a fatty acid salt content which ranges from 4% to 8 weight %.

5. The cosmetic product core of claim 1, wherein the basic core mass has a lipophilic material content that ranges between 5% to 40 weight %.

6. The cosmetic product core of claim 5, wherein the basic core mass has a fatty acid salt content which ranges from 10% to 35 weight %.

7. The cosmetic product core of claim 6, wherein the basic core mass has a fatty acid salt content which ranges from 16% to 32 weight %.

8. The cosmetic product core of claim 1, which comprises at least 60% to 80 weight % solid particles.

9. The cosmetic product core of claim 1, wherein the basic core mass comprises:

| | |
|---|---|
| fatty acid salt: | 1-20% |
| lipophilic substance | 5-40% |
| solid particles: | at least 50-90% |
| Additives: | 0-10% |

in which percentages are weight percentages.

10. The cosmetic product core of claim 1, wherein the core comprises the following composition:

| | |
|---|---|
| fatty acid salt: | 4-8% |
| lipophilic substance | 16-32% |
| solid particles: | 60-80% |
| Additives: | 0-10% |

wherein percentages are weight percentages.

11. The cosmetic product core of claim 1, wherein the fatty-acid salts contain 12 to 18 C atoms.

12. The cosmetic product core as defined in claim 11, wherein at least one fatty acid salt from the group consisting of calcium laureate, magnesium stearate, zinc stearate, and salts of palmitic acid, linoleic acid, oleic acid and myristine acid.

13. The cosmetic product core as defined in claim 11, wherein said at least one fatty acid salt is selected from the group consisting of lithium stearate, aluminum stearate and aluminum tristearate.

14. The cosmetic product core as defined in claim 11, wherein the fatty acid salt is aluminum distearate.

15. The cosmetic product core of claim 1, wherein said at least one lipophilic material is selected from the group consisting of mineral oil, castor oil, isopropylmyristate, isopropylpalmitrate, hydrogenated coconut oil, stearyldimethicone, capryl glyceride, caprin glyceride, hydrogenated palm-seed oil glycerides and hydrogenated palm-oil glycerides.

16. The cosmetic product core of claim 1, wherein the solid particles comprise inorganic and/or organic color pigments.

17. The cosmetic product core of claim 1, wherein the solid particles comprise particles selected from the group consisting of talcum, kaolin, mica, and mica coated with silicon dioxide.

18. The cosmetic product core of claim 1, further comprising at least one additive selected from the group consisting of aromatic substances, care substances, film-forming components, preservatives, sunscreen agents, perfumes, and vitamins.

19. A process for producing the cosmetic product core of claim 1, produced by extruding a basic starting mass, obtained by mixing together at least one fatty acid salt, at least one lipophilic substance with a melting point range<50° C. and 50% solid particles to form a basic mass; extruding the basic mass at a temperature ranging from 80-140° C.

20. A method of producing a cosmetic product selected from the group consisting of a lipstick, eyeliner, eye shadow, cosmetic pencil, rouge stick or other cosmetic stick, the method comprising: utilizing the cosmetic product core according to claim 1 to form the core of the cosmetic product.
